(19) United States
(12) Patent Application Publication
(10) Pub. No.: US 2012/0203141 A1
(43) Pub. Date: Aug. 9, 2012

(54) METHOD AND APPARATUS FOR REDUCING/SUPPRESSING PAIN IN DIGITS

(76) Inventors: Totada R. SHANTHA, McDonough, GA (US); Jessica G. SHANTHA, McDonough, GA (US); Erica M. SHANTHA, Stone Mountain, GA (US); Lauren A. SHANTHA, McDonough, GA (US); Usha MARTIN, Macon, GA (US)

(21) Appl. No.: 13/022,220

(22) Filed: Feb. 7, 2011

(51) Int. Cl. A61N 7/00; A61M 19/00; A61H 1/00

(52) U.S. Cl. 601/2; 601/46; 604/290

(57) ABSTRACT

A therapeutic device and method for blocking pain in a patient's digit includes a ring-like vibratory element adapted to be fitted about the digit temporarily and controller coupled to the ring-like vibratory element for controlling its operation. By applying vibration to the patient's digit, pain transmission is blocked or lessened along the digit, allowing the patient or a caregiver to provide treatment to the digit with reduced pain, such as when a diabetic's finger is to be stuck for drawing blood for a blood sugar measurement). Moreover, if the digit is injured, the present invention can provide pain relief from the injury as well. Further, the invention can be applied to the digit in combination with other pain-reducing therapies, such as the application of ice, the application of topical analgesics, the application of local anesthetics, etc. The controller includes an on/off control switch and a switch for controlling the vibratory frequency. The controller further includes a battery power source for powering the ring-like vibratory element. Also, the ring-like vibratory element includes an electrically-powered vibratory motor and/or a piezoelectric ultrasound transducer for creating vibrations in the digit. The vibratory element includes two vibratory parts, to be positioned on opposite sides of the digit, along the nerve bundles to maximize the blockage of pain.

### FIELD OF THE INVENTION

The invention relates to the treatment or prevention of pain, and in particular relates to the treatment and prevention of pain in the digits of the hands and feet.

### BACKGROUND OF THE INVENTION

Pain is the single most important reason for physician consultation in the United States, because it is a major symptom in countless diseases. Pain is experienced millions of times every day due to insertion of sharp needles to inject or draw fluids from the body. Many painful conditions resolve themselves rapidly once the stimulus that causes the pain is removed and the body begins to heal. Pain can persist after removal of the stimulus (vaccinations, needle stick) and sometimes pain arises in the absence of any detectable stimulus, damage or pathology. One of the most common painful experiences by children is a needle stick to the pulp of the finger to draw blood to test for sugar in diabetics, and needle stick during vaccinations. Millions of injections are given all over the world to administer therapeutic agents which results in pain, sometime unbearable.

An overarching fear of needles and needle sticks (lancing) exists by many people throughout the world. This fear knows no bounds of age, sex, or race. The effects of pain are remembered young and old alike. For many, this pain phobia makes visits to the doctor and dentist intolerable and oftentimes becomes a last resort or is totally ignored, resulting in a lack of care and the onset or worsening of conditions. The pain (or fear of pain) can also prevent the patient from testing every day (two to three times per day) for blood sugar levels in Type I diabetics.

Various approaches have been used in the past to reduce and/or eliminate needle sticking pain and the associated fear, without much success. One method of minimizing pain in a digit to be pricked for blood sugar measurement is to numb the finger with very cold water or ice. Unfortunately, dipping the finger in the cold water or applying the cold water or ice packs is not always practical, especially if one has to test for the blood sugar two to three times per day or take shots more than once per day. It is also known that one can apply a local anesthetic. However, application of a local anesthetic to reduce pain can take a long time. Another approach is to numb the digit by spraying with ethylene chloride to freeze the skin. However, this prevents the leaking and free flow of blood as a result in constriction of the digital blood vessels (BV), defeating the purpose of obtaining the blood samples. Spraying the skin with ethylene chloride is not suitable for vein punctures either. The nerve fasciculus that transmit the pain, cold and vibration sensation as well as other sensations from the digits and toes are bundled in two nerve fasciculi located at the 3 and 9 O'clock positions situated on the digital artery and vein bundle in the fingers. They are derived from the median (3½ fingers from thumb) nerve and ulnar nerve (1½ finger from little finger).

Lidocaine has long been used to decrease the pain of dental injections, with or without vibrating needles, but has not been found to be effective in other painful dermal needle sticking situations. Use of a simple handheld vibrating massager has been known to decrease injection pain in adults, but has not been known to have be effective for lancing the finger pulp and it has not been tried to induce anesthesia on the fingers. It is known that the application of ultrasonic/ultrasound and then the application of local anesthetic or vice-versa also enhances the penetration of local anesthetics, resulting in alleviation of pain from lancing and needle sticks.

The pulp of the finger is richly supplied with blood vessels and nerves, and is rich in Pacinian corpuscles and other pain carrying free nerve endings. These corpuscles are especially susceptible to vibrations, from which they can be stimulated even centimeters away (Kandel, Edited by Eric R.; Schwartz, James H.; Jessell, Thomas M. (2000), Principles of Neural Science, New York: McGraw-Hill, Health Professions Division). Their optimal sensitivity is 250 Hz and this is the frequency range generated upon finger tips by textures made of features smaller than 200 μm (Scheibert J, Leurent S, Prevost A, Debrégeas G. (2009). The Role of Fingerprints in the Coding of Tactile Information Probed with a Biomimetic Sensor. Science. 323(5920):1503-6).

The great sense of touch provided in human fingers makes needle sticks or lancing very painful, in large measure due to the rich sensory nerve network in the pulp of the finger. Therefore, there is a need for device to decrease the pain associated with finger pain pulp puncture for injection or accessing the BV.

### SUMMARY OF THE INVENTION

In a first preferred form the present invention comprises a therapeutic device for blocking pain in a patient's digit and includes a ring-like vibratory part adapted to be fitted about the digit temporarily. A controller is coupled to the ring-like vibratory part for controlling operation of the ring-like vibratory element. By applying vibration to the patient's digit, pain transmission is blocked or lessened along the digit, allowing the patient or a caregiver to provide treatment to the digit with reduced pain. For example, the invention can be applied to a finger prior to sticking the finger (as for drawing blood, for example, for a blood sugar measurement). Moreover, if the digit is injured, the present invention can provide pain relief from the injury as well. Further, the invention can be applied to the digit in combination with other pain-reducing therapies, such as the application of ice, the application of topical analgesics, the application of local anesthetics, etc.

The vibratory element can be selected or adapted to induce vibrations at different frequencies, as desired, including subsonic, sonic, and ultrasonic frequencies—ultrasound frequencies.

Optionally, the ring-like vibratory element includes a first section and a second section hingedly coupled to the first section at first ends thereof. Preferably, the first and second sections of the ring-like vibratory element are each generally C-shaped. Preferably, an openable closure is provided for selectively securing second ends of the first and second sections to one another. In one form, the openable closure comprises a hook and loop fastener for releasably securing the second ends of the first and second sections to one another.

Optionally, the ring-like element can include an inner elastic loop instead of a vibratory element to be slipped around a digit and one or more vibratory motors mounted to the outside of the inner elastic loop.

Optionally, an elastic element can be provided for elastically securing second ends of the first and second sections to one another. Additionally, a second elastic element can be provided so that both the first and second ends of each section are elastically secured to one another. Furthermore, a clasp-like element can be provided for releasably securing second ends of the first and second sections to one another.

Optionally, the vibrations can be ultrasonic or a combination of ultrasonic and non-ultrasonic vibrations.

Optionally, the controller includes an on/off control switch and a switch for controlling the vibratory frequency. Preferably, the controller includes a battery power source for powering the ring-like vibratory element. Also preferably, the ring-like vibratory element includes an electrically-powered vibratory motor for creating vibrations in the digit.

The ring-like vibratory element can be adapted to apply vibrations (including ultrasonic) to an entirely circumferential region of the digit (all the way around the digit) or can be adapted to apply vibrations to just selected regions of the digit. In this regard, it is noted that typically the nerve bundle extending within and along the digit tend to be positioned in specific, known locations such that the ring-like vibratory element can be adapted to apply vibratory energy adjacent the nerve bundles, while leaving other portions of the digit be as is. In other words, the ring-like vibratory element can be adapted to target the nerves.

One good example of a ready application for the present invention is a digital vibrator to reduce needle or lancing pain at the finger, thumb, or toes, such as when used to get a blood sample to test for blood sugar levels and other laboratory tests. This is particularly helpful for diabetics in that they often have to stick themselves in the digits repeatedly, often several times per day. Many diabetics find this painful and have to endure such pain many times per day. The present invention can provide substantial relief to such patients, for example.

The present invention can also be used in conjunction with a local anesthetic port patch to enhance the uptake of local anesthetics from the patch from the surface of the skin. The vibrator can also induce numbness on its own.

In another preferred form the invention comprises a method for blocking pain in a patient's digit and includes the steps of: (a) securing a pair of vibratory and/or ultrasound—ultrasonic elements to the patient's digit, positioned on opposite sections of the digit along the nerve bundles, the vibratory elements adapted and sized for vibrationally stimulating opposite sides of a patient's digit; and (b) operating the vibratory and/or ultrasonic elements by a controller coupled to the vibratory elements.

Preferably, these steps (a, b) are carried out prior to a needle stick in the patient's digit. Also preferably, before the needle stick takes place, one applies a local anesthetic to a portion of the patient's digit to be subjected to the needle stick. In one preferred form, the step of applying a local anesthetic comprises applying a transdermal patch to the patient's digit, the patch being adapted to deliver an anesthetic locally to the digit.

These methods combined will have augmentation—amplification effects to relieve the pain of needle sticks for any and all therapeutic applications such as injection of therapeutic agents and vein or arterial access for any and all medical applications.

Such a device and method according the invention is reusable, practical, easy to clean, usable in all age groups, can operate with a rechargeable or disposable battery (or batteries), and is not be cumbersome to use. Moreover, the digital vibration device (DVD) described herein can be used with or without the use of a transdermal local anesthetic patch port. Furthermore, the digital vibration device can be used with a local anesthetic band aid. It can also be used for these needs and others to take away pain in situations such as removing splinter, burns, or injury to fingers, nails, any and all painful conditions that the present invention is aimed at.

### BRIEF DESCRIPTION OF THE DRAWINGS

FIG. 1 is a schematic illustration of a therapeutic device for blocking pain in a patient's digit according to a first preferred form of the invention, showing a ring-like vibratory part and a control box/power supply.

FIG. 2 is a schematic illustration of a therapeutic device for blocking pain in a patient's digit according to another preferred form of the invention, showing a ring-like vibratory part and omitting a control box/power supply for simplicity of illustration.

FIG. 3 is a schematic illustration of a therapeutic device for blocking pain in a patient's digit according to another preferred form of the invention, showing a ring-like vibratory part and omitting a control box/power supply for simplicity of illustration.

FIG. 4 is a schematic illustration of a therapeutic device for blocking pain in a patient's digit according to another preferred form of the invention, showing a ring-like vibratory part and omitting a control box/power supply for simplicity of illustration.

FIG. 5 is a schematic illustration of a therapeutic device for blocking pain in a patient's digit according to another preferred form of the invention, showing a ring-like vibratory part and a control box/power supply.

FIG. 6 is a schematic illustration of the therapeutic device of FIG. 5 for blocking pain in a patient's digit, showing a ring-like vibratory part positioned about a patient's digit and showing a control box/power supply in the palm of the patient's hand, and shown in conjunction with an optional anesthetic port band aid positioned toward a distal end on the digit.

FIG. 7 is a schematic illustration of an optional anesthetic port band aid (transdermal patch) as shown in FIG. 6.

FIG. 8 is a schematic cross-section of a human digit, in particular a finger, and showing the anatomy and histology of the finger.

### DETAILED DESCRIPTION OF THE INVENTION

The embodiments of the invention used to produce local analgesic, and local anesthesia devices and methods are now disclosed with or without the use of previously described the transdermal local anesthetic patch port. The term needle or lancet referrers to any hypodermic needle or thin lace provided for lancing the pulp of the finger to draw blood especially in diabetics.

The injection needle or Hypodermic needles as used herein includes, but is not limited to, all different types of sharp pointed needles, pins, probes, sharp instruments and the like that can be used to inject therapeutic agents; remove, or introduce fluid medications; lance, incise, evacuate tissues of the body, and the like to a patient, as well as to conduct tests such as but not limited to allergy tests to a patient and inject daily hormones such as Insulin in diabetics and other daily, biweekly, thrice weekly injections for health maintenance or as therapeutic agents to treat diseases.

The term transdermal local anesthetic patch port used herein includes, but is not limited to, the use or application of local anesthetic to the skin and mucous surfaces on any part of the body a person or a patient to induce an analgesic or anesthetic effect.

The term “Vibration, vibrational, vibromatic, sonovibramtic, analgesia” as used herein includes, but is not limited to, the use or application of vibration and sound (including ultrasonic from the digital vibration device (DVD) to a patient to induce an analgesic or anesthetic effects of the selected area including the digits for introduction of needle or lancet through the pain sensitive dermis or mucous membrane.

“Vibrational and the transdermal local anesthetic patch port analgesia” as used herein includes, but is not limited to, the use or application of both the transdermal local anesthetic patch port in conjunction with the application of vibration with the help of the VD to a patient digits, skin or mucous membrane surface to induce an local analgesic or anesthetic effect to facilitate the entry of a lancet or needle or to be use as therapeutic method to relive pain due multiple etiological factors.

The term “Oculopathies” used herein includes, but is not limited to, the use or application various painful and non painful diseases of the eyes with or without therapeutic agents.

The present invention has ready application to decreasing pain, such as that related with needle sticks (venous accesses or injection of therapeutic agents) or lancing of the finger pulp to draw blood for testing glucose, electrolytes, cholesterol, hemoglobin hematocrit and many other laboratory diagnostic and therapeutic tests.

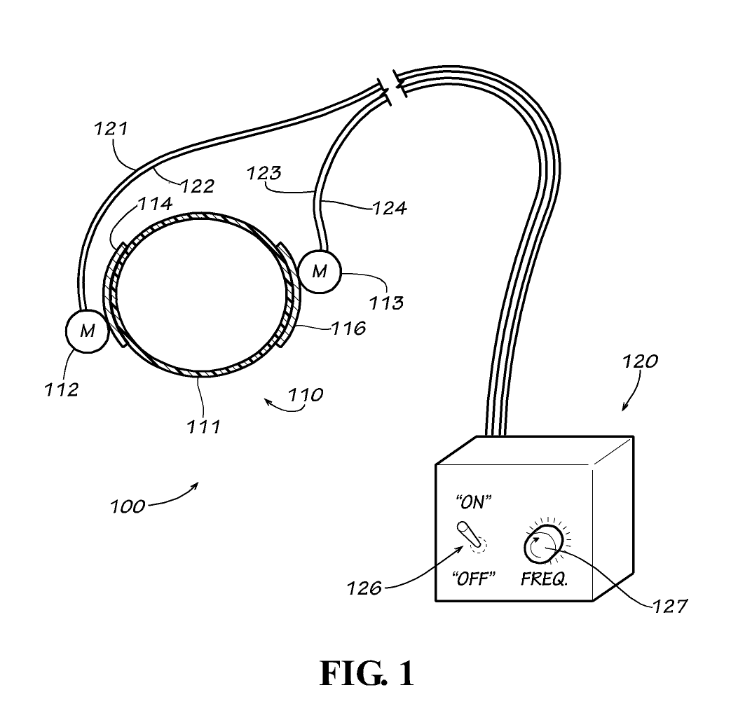

FIG. 1 is a schematic illustration (not to scale) of a therapeutic device 100 for blocking pain in a patient's digit according to a first preferred form of the invention, showing a ring-like vibratory part 110 and a control box/power supply 120. The ring-like vibratory part 110 is adapted to be fitted about the patient's digit temporarily. The controller or control box 120 is coupled to the ring-like vibratory part for controlling operation of the ring-like vibratory element 110 with electrical wires, here two pairs of wires 121-124. By applying vibration to the patient's digit, pain transmission is blocked or lessened along the digit, allowing the patient or a caregiver to provide treatment to the digit with reduced pain. The vibratory element 110 and the controller 120 can be selected or adapted to induce vibrations at different frequencies, as desired, including subsonic, sonic, and ultrasonic frequencies.

In this first example form shown, the ring-like vibratory element 110 can include an inner elastic loop 111 adapted to be slipped around a digit and one or more vibratory motors 112, 113 mounted to the outside of the inner elastic loop. As shown, thin metal strips 114, 116 are bonded to the elastic loop for supporting the vibratory motors and transmitting vibrations to the thin elastic loop.

Optionally, the controller 120 includes an on/off control switch 126 and a rotary switch 127 for controlling the vibratory frequency. Preferably, the controller 120 includes a battery power source for powering the ring-like vibratory element. Alternatively, instead of using a battery to power the controller 120, the controller can be provided with a 120 VAC plug for plugging into a wall outlet. Typically, the motors 112, 113 will be DC motors such that the controller 120 would then have an AC-to-DC converter to supply the vibratory elements with DC current.

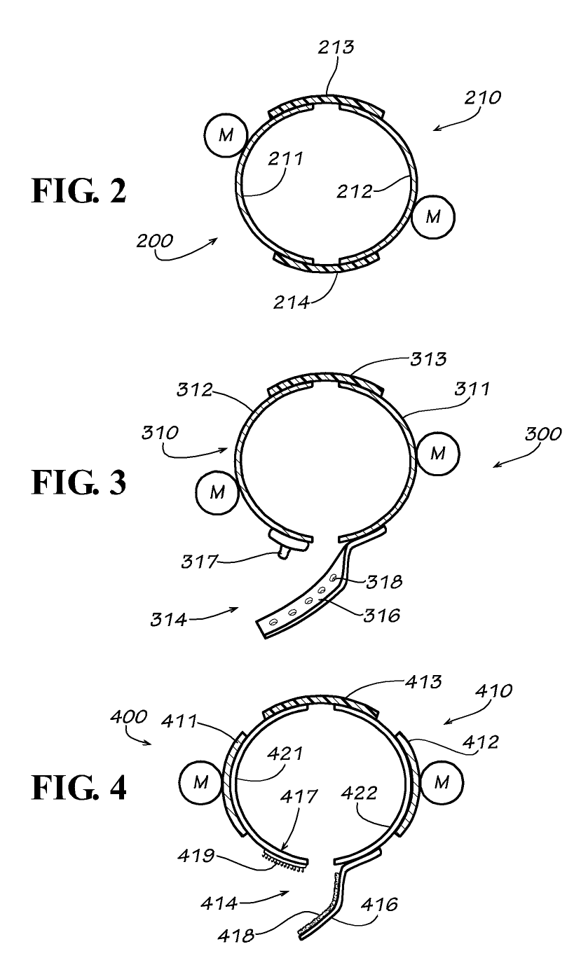

FIG. 2 is a schematic illustration of a therapeutic device 200 for blocking pain in a patient's digit according to another preferred form of the invention, showing a ring-like vibratory part and omitting a control box/power supply for simplicity of illustration. In this embodiment, the controller is as above, so its description is not repeated herein. In this embodiment, the ring-like vibratory element 210 includes two arcuate metal strips 211, 212 coupled together by a pair of flexible elements 213, 214. The flexible elements 213, 214 can be rubber strips as depicted or can be fabric. Elasticity in these elements, along with some torsional flexibility, will facilitate the mounting of the ring-like vibratory part 210 over the patient's digit (finger or toe). In this regard, the elasticity allows the device to be slipped over the digit by simply stretching action. As above, the metal strips 211, 212 bear vibratory motors that impart vibration to the metal strips.

FIG. 3 is a schematic illustration of a therapeutic device 300 for blocking pain in a patient's digit according to another preferred form of the invention, showing a ring-like vibratory part 310 and omitting a control box/power supply for simplicity of illustration. In this embodiment, the ring-like vibratory element 310 includes two arcuate metal strips 311, 312 coupled together by a flexible element 313 and a closure 314. The flexible element 313 acts as a sort of hinge and can be a rubber strip as depicted or can be fabric. The closure 314 is in the form of a hasp, much like a buckle and includes a flexible elongate portion 316 bonded to the metal strip 311 and a post-like portion 317 bonded to the metal strip 312. The flexible elongate portion has a series of perforations, such as perforation 318, formed therein for receiving the post therethrough at different positions, thereby allowing for some adjustability in the size of the digit to be encircled. To place the device on a digit, one opens the closure 314 and opens the ring-like element and slips the device over the digit. Once over the digit, one can close the closure by engaging the post through one of the perforations. As above, the metal strips 311, 312 bear vibratory motors that impart vibration to the metal strips.

FIG. 4 is a schematic illustration of a therapeutic device 400 for blocking pain in a patient's digit according to another preferred form of the invention, showing a ring-like vibratory part 410 and omitting a control box/power supply for simplicity of illustration. In this embodiment, the ring-like vibratory element 410 includes two arcuate metal strips 411, 412 coupled together by a flexible element 413 and a closure 414. The flexible element 413 acts as a sort of hinge and can be a rubber strip as depicted or can be fabric. The closure 414 is in the form of a hasp, much like a buckle and includes a flexible elongate portion 416 with one end bonded to the metal strip 412 and bearing hook and look fastener material 418 on a distal end. The closure 414 also includes a portion 417 bonded to the metal strip 411 and bearing hook and loop fastener material 419. The hook and loop fastener material can be attached to each other at different positions, thereby allowing for some adjustability in the size of the digit to be encircled. To place the device on a digit, one opens the closure 414 and opens the ring-like element and slips the device over the digit. Once over the digit, one can close the closure by engaging the post through one of the perforations.

Moreover, as shown in FIG. 4, an optional fabric or resilient liner 421, 422 can be attached to the inside of the metal strips 411, 412 to make the device more comfortable.

The ring-like vibratory elements described above can be adapted to apply vibrations to an entirely circumferential region of the digit (all the way around the digit) or can be adapted to apply vibrations to just selected regions of the digit. In this regard, it is noted that typically the nerve bundle extending within and along the digit tend to be positioned in specific, known locations such that the ring-like vibratory element can be adapted to apply vibratory energy adjacent the nerve bundles, while leaving other portions of the digit alone. In other words, the ring-like vibratory element can be adapted to target the nerves, as desired. In this regard, the split designs depicted herein showing two arcuate (C-shaped) metal strips are preferred as an effective way to target the nerve bundles in the digit.

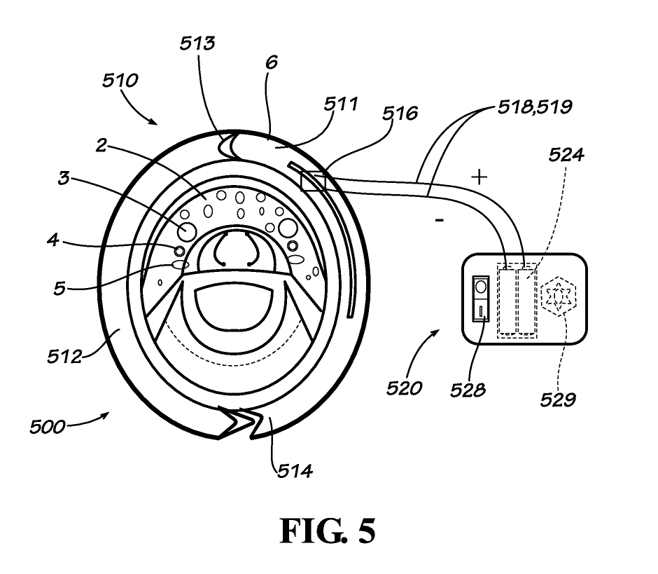

FIG. 5 is a schematic illustration of a therapeutic device 500 for blocking pain in a patient's digit according to another preferred form of the invention, showing a ring-like vibratory part 510 and a control box/power supply 520. FIG. 5 (as does FIG. 8) also shows the anatomy and histology of the finger cross section 100 showing the pulp of the finger which contains rich arteriovenous plexus and vast network of pain nerve fibers, where the lancing is performed to extract drops of blood to test for blood sugar and other laboratory parameters to maintain health and test for any physiological abnormality for diagnosis of disease. The diagram shows the pulp 2 of the fingers with rich BV plexus (circles) and nerves (black dots), digital nerve fasciculus 3 which supplies the pulp; digital artery 4 and digital vein 5 which join with each other at the pulp of the terminal digits to form rich vascular network. Pulp contains a rich network of terminal sensory nerve fibers and nerve endings which convey the pain, pressure, vibration, heat and cold sensations to the spinal cord and then to the central nervous system, such as due to penetration of a needle or lancets. It is from these blood vessels the blood is drawn by after lancing the pulp of the digits.

Moreover, as shown in FIG. 5, the digital (relating to anatomical digits, not digital as in digital electronics) vibrator device (DVD) 510 comprises a ring-like element to be wrapped around a digit, such as a finger or toe. As shown herein, the digital vibratory device includes a first C-shaped portion 511 hinged to a second C-shaped portion 512 at a hinge 513. A hook and loop fastener closure 514 is positioned opposite the hinge 513 and releasably secures ends of the C-shaped portions 511, 512 to each other.

Vibration is imparted in the ring-like element 510 by a vibrational motor 516 connected by power cables 518, 519 to a control box 520. The vibratory motor 516 can be activated to deliver 300 to 6000 vibrations per minute, according to the need and tolerability of the patient. The vibrations can be adjusted to the comfort level of the patient to obtain the maximum pain relief within a minimum time. The vibration can be set, for example, to 300, 500, 750, 1000, 1500, 2000, 3000, 4000, 5000 or 6000 vibrations per minute. Of course, these frequencies are just examples and other frequencies can be chosen. The vibration motor 516 can be switched between vibration cycles by a potentiostatic switch 529 or other mechanism. The vibration motor 516 can be varied continuously or step-wise between 300 vibrations per minute and 6000 vibrations per minute using the potentiostatic switch 529. The power to operate the vibrator is provided by the battery pack 524 connected by positive and negative wires. The power switch 528 to turn on the vibrator is located on the housing of the control box 520 (also can be referred to as a battery pack).

The power source 524 can be any of well-known battery technologies, including conventional alkaline, NiCad, lithium ion, or nickel metal hydride batteries or other battery types. The batteries or battery can be rechargeable or not, as desired. Moreover, instead of using a battery, a continuous source of electric power can be employed, such as 120 VAC from wall outlet. If a rechargeable energy pack is used, optionally one may also include include a recharger base.

Switch 528 can be a regular, ordinary on/off switch, such as a toggle, lever, push-button, capacitance or other switch. Switch 529 can be eliminated and instead its function can be automatically incorporated in the control box which would then vary the vibration cycle of the motor. The switch 529 can be implemented as a common potentiostat. This category of control switch 529 would be functional with a vibration motor that operates at many different vibration cycles.

Notice that the vibrator can be slipped on the digit as a ring or through the hinge arrangement 513 to open and attach the ring-like vibratory element to the digit. If the device is to be used to lessen or block pain for the lancing of a fingertip, as is common in blood sugar measurement, the device can conveniently be placed approximately an inch to an inch and a half from the very tip of the finger. The hook and loop fastener 514 holds the vibrator tightly in place.

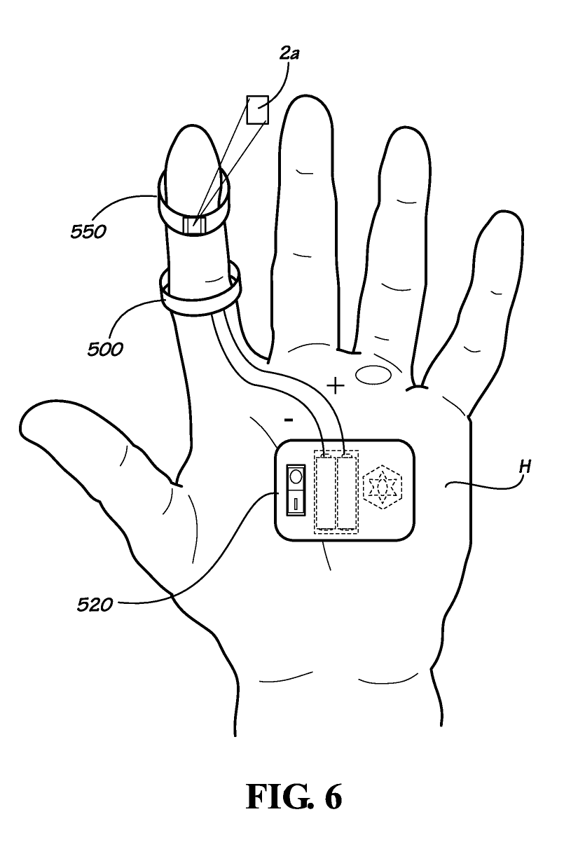

FIG. 6 shows a DVD vibrator 500 placed on a digit of the hand H and local anesthetic port band aid 550 on the digit with the site to be lanced 2a. It shows the application of the transdermal local anesthetic patch port band aid 550 around the finger pulp with lancet entering the area 2a at the anesthetized band aid patch port. The lancet or needle is puncturing through this transdermal local anesthetic patch port to draw the blood. After drawing the blood, one need not have to apply a new band aid. This band aid 300 seals the skin puncture, maintaining sterility, and keeps the finger tip pain free for long time after the insult. The finger terminal pulp site can be accessed four times without pain with the transdermal local anesthetic patch port. The digital vibration device 500 (DVD) can be applied proximal to the transdermal local anesthetic patch 550, with controls of the device 520 placed in the palm of the hand. This can be activated for a minute or two or five or any length of time to obtain the maximum analgesia and anesthesia as the terminal digital pulp is accessed at 2a. The digital vibration devices 100, 200, 300, 400, 500, described above, can be used as shown in this figure.

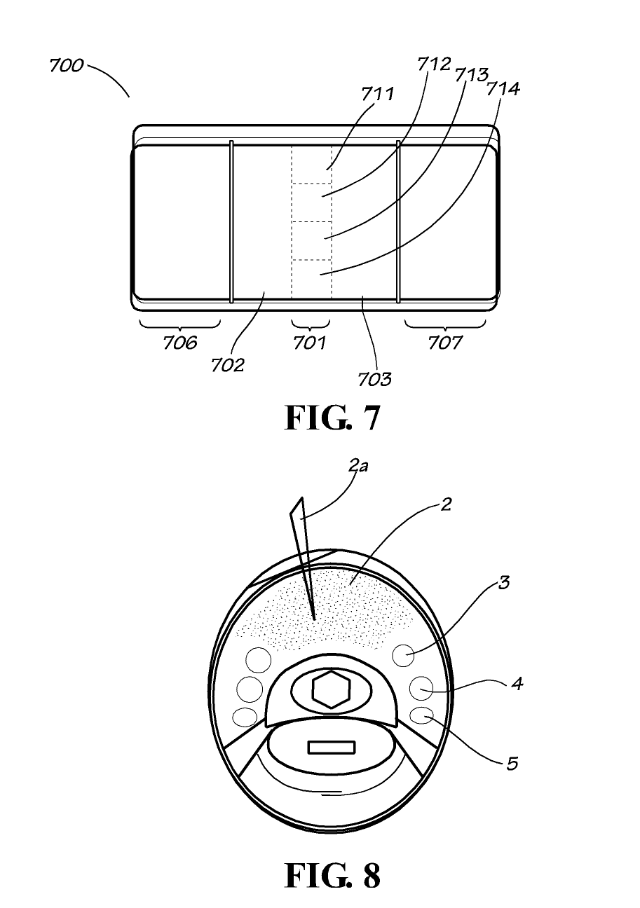

FIG. 7 is a schematic diagram of a transdermal local anesthetic patch (a port band aid) 700 and shows the underside of thereof (i.e., the side that is applied to the skin). The patch 700 includes a central region 701 flanked by two intermediate regions 702, 703, and two outer regions 706, 707. The central region 701 includes four resealable injection ports 711-714. In this respect, anesthetic reservoirs are formed in the intermediate regions 702, 703 and are adjacent to the injection ports 711-714 (flanking the injection ports). An adhesive can be applied to the surfaces in the outer regions 706, 707, which are composed of an impermeable material. Although not shown in this figure, a protective cover can be applied to the underside of the transdermal local anesthetic patch 700 (and any of the other devices described herein) to prevent the anesthetic from leaking, as well as preventing contamination of the injection port. The protective cover can be readily peeled off prior to use. An adhesive can be applied to the surfaces which are composed of an impermeable material and can be easily peeled off.

FIG. 8 is a schematic cross-section of a human digit, in particular a finger, and showing the anatomy and histology of the finger. Advantageously, the present invention takes advantage of the positions of the digital nerve 3, artery 4, and vein 5 in the finger, located at the 3 and 9 o'clock positions where the maximum vibration is concentrated from the DVD.

The transdermal local anesthetic patch port and the digital vibration device (DVD) can be used to treat various painful conditions of the fingers, phalanges, nails, etc, including trauma, infection, fractures, tendon tears, burns, allergic reactions, any and all painful conditions of the digits, toes and nails of the hand and leg besides relieving the pain of lancing the pulp of the digits to draw blood.

The present invention is believed to helps to reduce pain and induce analgesia based on the “gate” theory of pain. Based on gate theory, vibration helps to reduce pain as the vibration of cutaneous nerves send impulse to the spinal cord gate and block the gate and prevents the higher center receiving the pain impulses. The transdermal local anesthetic patch port on the other hand works differently, and its analgesic—anesthetic effects are local. It stops pain by blocking the sodium and potassium channels needed to generate nerve impulse. It is local and entirely different mechanism form the vibration method of inducing analgesia. That is why, the pain relief by using a vibration device is analgesic, meaning reducing the feeling the sensation of pain, not complete anesthesia as seen in the transdermal local anesthetic patch port.

The theory behind the pain relief of the present invention is believed to be consistent with Melzack and Wall's gating mechanism within the spinal cords. The pain gate is closed in response to normal stimulation of the fast conducting “touch” possibly vibration nerve fibers (vibration stimulation); but opened when the slow conducting “pain” fibers transmitted a high volume and intensity of sensory signals. The gate could be closed again if these signals were countered by renewed stimulation of the large fibers (Ronald Melzack and Patrick Wall “Pain Mechanisms: A New Theory,” Science: 150, 171-179, 1965). Those authors proposed that thin (“pain”) and large diameter (“touch, pressure, vibration”) nerve fibers carry information from the site of injury to two targets in the dorsal horn of the spinal cord: the “inhibitory” cells and the “transmission” cells. Signals from thin and large diameter fibers excite the transmission cells, and when the output of the transmission cells exceeds a critical level, pain begins. The job of the inhibitory cells is to inhibit activation of the transmission cells and shut the gate. When thin (pain) and large (touch, etc) fibers, activated by a noxious event (lancing, needle sticks), excite a spinal cord transmission cell, they also act on its inhibitory cells. The thin fibers impede the inhibitory cells (tending to leave the gate open) while the large diameter fibers excite the inhibitory cells (tending to close the gate-vibration sense). So, the more large fiber activity relative to thin fiber activity coming from the inhibitory cell's receptive field, the less pain is felt. Vibration induces analgesia by partially closing the gate through the large fibers.

Pain signals can also travel from the site of injury directly up the cord to the brain (bypassing the inhibitory and transmission cells) where, depending on the state of the brain, it may trigger a signal back down the spinal cord to modulate inhibitory cell activity (and so pain intensity). This was the first theory to offer a physiological explanation for the previously reported effect of psychology on pain perception. Perception and blocking of the pain is very complicated. The vibration activates large nerve fibers first, then small pain fibers, thus inhibiting the pain at the site of lancing or injection by shutting the pain gate in the spinal cord. It is only partial closure that is why there is analgesia, not complete anesthesia as seen by use of a transdermal local anesthetic patch port. That is why the combination used of a vibratory device and a transdermal local anesthetic patch port is effective in relieving the acute pain due to needle stick of any kind and lancing effectively in rapid succession.

The present invention may possibly be used in treatment of many acute, sub-acute, and chronic painful conditions of the fingers, thumb, nails, and toes. The pain can be due to stings, burns, scratches, splinters, bruises, contusions, scratches, blunt trauma, localized arthritis's pain, dislocation, stretching, twisting of fingers, digital fracture manipulation, tendon tears, and like conditions. The finger pain can also be resulting from the effect systemic diseases.

Our invention of DVD, alone or in conjunction with a transdermal local anesthetic patch port, can be used to lance the pulp of the digits to obtain drops of blood, vein puncture and other method where the needle is introduced into the body. Moreover, it is also useful in the treatment of various other painful conditions.

Optionally, a small battery-powered ultrasound transducer can be provided for producing a low-frequency (approximately 20 kHz) ultrasonic energy to the skin for 20 to 120 seconds, preferably adjacent the transdermal patch port. It can be used before the application of a transdermal patch or after the application of the transdermal patch with local anesthetics. The ultrasound waves open small cavities in the skin by disorganizing the lipid bi-layer, creating tiny, reversible channels through which the local anesthetics, anti-microbial and other therapeutic agents are delivered to the deeper layers of the skin. The ultrasound also disrupt and break open the microbes at the site of injection, thus reducing the chances of infection of the site of injection and/or the microbes being carried to the deeper layers of the skin. Further, the antibiotics, and antibacterial and antiviral therapeutic agents become more effective in keeping the injection patch port sterile. The microscopic openings created in the skin by ultrasound are much too small to see, but large enough for molecules relevant to medicine to pass through the skin and mucous membranes.

The optional ultrasound transducer can be in the form of a piezoelectric transducer, which when excited by electricity, vibrates at frequencies above sound. Such a transducer can be used in place of the vibratory motors described herein or can be used in conjunction therewith. The transducer can be a piezoelectric crystal, ceramic, polymer, or composite. Typically, such transducers tend to operate at a harmonic frequency at a fixed frequency that is directly related to the thickness of the transducer (hence, for many ultrasound transducers, the frequency of the harmonic vibrations is not variable, typically). The electric circuit and control for such a transducer can take advantage of this harmonic tendency by driving the transducer at a sympathetic (matching) frequency. Thus, the circuit is designed to match the harmonics of the transducer and drive the transducer with an oscillating signal that matches the harmonic frequency of the transducer. Of course, other circuit designs can be employed, as desired, and this is merely one example. In addition, the ultrasound transducer is driven to be either off or on and the circuit needs an on/off switch of some sort and an appropriate power supply.

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection. This is presently deemed to be the most practical and preferred embodiments of the invention. The invention will be apparent to those of ordinary skill in the art that numerous modifications, including, but not limited to, variations in size, materials, shape, form function, and manner of procedure, assembly, and the use may be made. The preferred embodiment of the present invention has been described. The invention should be understood that various changes, adaptations, and modifications may be made thereto. It should be understood, therefore, that the invention is not limited to details of the illustrated invention. This method can be used to treat all surface painful conditions and as well as prevent them when the injection needle or lancet is introduced through the skin. The preferred embodiment of the present invention has been described. The invention should be understood that various changes, adaptations and modifications may be made thereto. This should be understood, therefore, that the invention is not limited to details of the illustrated invention examples.

1. A therapeutic device for blocking pain in a patient's digit, the device comprising:
a ring sized and configured to be placed around the patient's digit and housing a pair of vibratory elements, the ring being adapted to be fitted about the patient's digit temporarily, wherein the vibratory elements comprise a first curved section and a second curved section hingedly coupled to the first curved section at first ends thereof; and
a controller electrically coupled to the vibratory elements for controlling operation of the vibratory elements.

2. (canceled)

3. A therapeutic device for blocking pain as claimed in claim 1 wherein the first and second curved sections are each generally C-shaped.

4. A therapeutic device for blocking pain as claimed in claim 1 wherein the first and second sections further comprise second ends and further comprising an openable closure for selectively securing the second ends of the first and second sections to one another.

5. A therapeutic device for blocking pain as claimed in claim 4 wherein the openable closure comprises a hook and loop fastener for releasably securing the second ends of the first and second sections to one another.

6. A therapeutic device for blocking pain as claimed in claim 1 wherein the first and second sections further comprise second ends and further comprising an elastic element for elastically securing the second ends of the first and second sections to one another.

7. A therapeutic device for blocking pain as claimed in claim 1 wherein the first and second sections further comprise seconds ends and further comprising a clasp-like element for releasably securing the second ends of the first and second sections to one another.

8. A therapeutic device for blocking pain as claimed in claim 1 further comprising first and second elastic elements for elastically securing the first and second sections to one another.

9. A therapeutic device for blocking pain as claimed in claim 1 wherein the first and second sections further comprise seconds ends and further comprising an elastic element for elastically securing the second ends of the first and second sections to one another.

10. A therapeutic device for blocking pain as claimed in claim 1 wherein the ring includes an inner elastic loop adapted to be slipped around a digit and wherein one or more vibratory motors are mounted to the inner elastic loop.

11. A therapeutic device for blocking pain as claimed in claim 1 wherein the vibratory elements comprises one or more ultrasonic devices.

12. (canceled)

13. (canceled)

14. (canceled)

15. A method for blocking pain in a patient's digit prior to a needle stick in the patient's digit, the method comprising steps of:
a) prior to a needle stick in the patient's digit, securing a pair of vibratory elements to the patient's digit, positioned on opposite sections of the digit along the nerve bundles, the vibratory elements adapted and sized for vibrationally stimulating opposite sides of a patient's digit;
b) operating the vibratory elements by a controller coupled to the vibratory elements
c) before or after step (a) above, applying a local anesthetic to a portion of the patient's digit to be subjected to the needle stick before the needle stick.

16. (canceled)

17. (canceled)

18. The method for blocking pain in a patient's digit as claimed in claim 15 wherein the step of applying a local anesthetic comprises applying a transdermal patch to the patient's digit, the patch being adapted to deliver an anesthetic locally to the digit.
